(19) United States
(12) Patent Application Publication
(10) Pub. No.: US 2001/0039262 A1
(43) Pub. Date: Nov. 8, 2001

(54) Methods and compositions for the treatment of cardiac indications

(76) Inventor: Balaji Venkataraman, Roswell, GA (US)

Correspondence Address: Cheryl L. Huseman, Esq., KILPATRICK STOCKTON LLP, 2400 Monarch Tower, 3424 Peachtree Road, N.E., Atlanta, GA, 30326, US

(21) Appl. No.: 09/842,887

(22) Filed: Apr. 26, 2001

Related U.S. Application Data
(60) Provisional application No. 60/200,157, filed on Apr. 26, 2000.

(51) Int. Cl. A61K 38/05; A61K 31/365

(52) U.S. Cl. 514/19; 514/462

(57) ABSTRACT

The present invention is directed to methods and compositions for treating cardiac indications, such as heart failure, hypertension and congestive heart failure, in an animal by administration of compositions comprising combinations of at least two or more agents into a single administrative dose. Such compositions and methods provide a means to treat cardiac indications with several agents with out administering multiple individual agents either concurrently or separately. Methods and compositions comprising combinations of at least two or more agents provide a way to administer multiple agents that is easier for patients to use, is more easily administered by caregivers, and that facilitates the physicians determination of drug interactions, patient side effects and dosages.

### CROSS-REFERENCE TO RELATED APPLICATIONS

This application claims priority to U.S. Provisional Application Ser. No. 60/200,157 filed on Apr. 26, 2000.

### TECHNICAL FIELD

The present invention is directed to methods and compositions for the treatment of cardiac indications such as hypertension, heart failure, and congestive heart failure. More particularly the invention relates to compositions comprising at least two or more agents for the treatment of cardiac indications, wherein the agents are provided in a single dministrative dose.

### BACKGROUND OF THE INVENTION

One of the more common cardiac indications in humans and other animals is heart failure (HF), a clinical condition resulting from failure of the heart to maintain adequate circulation of blood. Causes of heart failure include hypertension, infections, pericardial effusion, valvular insufficiency, coronary disease, congenital malformations, arteriosclerosis, constrictive pericarditis, atherosclerosis and hyperthyroidism.

Hypertention is a condition in which a patient has higher than normal blood pressure. The primary factor in hypertension is an increase in peripheral resistance resulting from vasoconstriction or narrowing of peripheral blood vessels. There are various causes of hypertension such as coarctation of the aorta, hyperthyroidism, arteriovenus fistula, and certain forms of renal disease. Hypertension often leads to heart failure, including congestive heart failure.

Congestive Heart Failure (CHF) is the symptomatic myocardial dysfunction resulting from heart failure and reduced outflow of blood from the heart. It has been estimated that more than 2 million patients in the United States suffer from heart failure and about 400,000 new patients develop CHF each year. There is an annual mortality rate of 40-50% in patients with severe heart failure. Approximately half the deaths in patients with CHF are secondary to the progression of cardiac insufficiency and its associated conditions. The remaining patients die from sudden cardiac death, which is presumably related to electrical instability and ventricular arryhythmias. Studies in patients with CHF indicate that heart failure is a progressively deteriorating condition with 20-40% of patients dying within 5 years after the onset of illness.

The function of the heart is to pump adequate blood to various organs in the body to furnish oxygen and substrates and to remove metabolites. In the normally functioning heart, the ventricles contract and force blood out during systole. This is followed by diastole, wherein the muscle fibers of the heart lengthen, the heart dilates and fills with blood. Heart failure occurs when ventricular contraction is compromised and the heart cannot meet the prevailing demand for blood. Heart failure is generally characterized by an inadequacy of the heart to meet the metabolic demands of peripheral organs and tissues either at rest or during stress.

In many forms of heart disease, the clinical manifestations of HF may reflect impairment of the left or right ventricle and systolic or diastolic dysfunction, or combined systolic and diastolic abnormalities. Whether the failure is primarily systolic or diastolic and regardless of which ventricle is affected, various hemodynamic, renal, and neurohumoral responses may occur. Left ventricular (LV) failure characteristically develops in coronary artery disease, hypertension, and most forms of cardiomyopathy. Right ventricular (RV) failure is most commonly caused by prior LV failure and tricuspid regurgitation. In systolic dysfunction, the heart fails to provide tissues with adequate circulatory output and is commonly caused by coronary artery disease, hypertension and dilated congestive cardiomyopathy. Diastolic dysfunction accounts for 20 to 40% of cases of HF and is presumed to be dominant in hypertrophic cardiomyopathy, hypertension, advanced aortic stenosis, and amyloid infiltration of the myocardium.

Congestive heart failure is characterized by venous stasis and reduced outflow of blood from the heart. There are typical hemodynamic, renal, and neurohumoral responses, characterized by symptoms such as weakness, breathlessness, abdominal discomfort, and edema in the lower portions of the body. Regardless of its etiology, in congestive heart failure there is a weakness of the myocardial tissue of the left and/or right ventricle of the heart and diminishing ability to pump and circulate blood into systemic and pulmonary circulation systems. If left untreated, the health of a patient with CHF could progress to the point where the disease would be fatal.

Heart failure, and in particular congestive heart failure, is currently treated with a combination of several individual agents, such as ACE inhibitors and diuretics. Treatment with diuretics provides effective symptomatic relief of moderate to severe congestive symptoms of HF resulting from venous stasis and reduced outflow of blood. The agents improve symptoms and functional capacity by promoting excretion of sodium and water, and helping to lower the plasma volume, which reduces congestion in the pulmonary and systemic vascular systems. A reduction in atrial and ventricular diastolic pressures relieves stress on the ventricular wall and promotes subendocardial perfusion. Diuretics may improve ventricular function even in asymptomatic patients. Two types of diuretics, loop diuretics and thiazides, are most commonly used to reduce fluid retention in patients with HF. A third group of diuretics, potassium sparing diuretics, are also useful in managing cardiac indications such as heart failure.

Because different diuretics provide different approaches to treating patients, the choice of agent is dependent upon the patient and the clinical state. Loop diuretics are considered safer and may provide better patient response than thiazide diuretics and are often more effective in patients with advanced symptoms of CHF. Acting on the ascending Loop of Henle in the kidney, loop diuretics can inhibit the reabsorption of as much as 25% of the glomerular filtrate and they are extremely efficacious at low doses. The most commonly used loop diuretics are ethacrynic acid, furosemide and bumetanide. Furosemide inhibits the reabsorption of sodium and chloride by the thick ascending limb by competing with chloride for a binding site on the Na+, K+, 2Cl−cotransporter.

Treatment with potassium sparing diuretics decrease active sodium reabsorption and potassium excretion. Potassium sparing diuretics are ordinarily used in combination with thiazides or loop diuretics to restrict potassium losses and sometimes augment diuretic action. Potassium sparing diuretics comprise 3 pharmacologically distinct groups: aldosterone antagonists, pteridines, and pyrazinoylguanidines. The site of action of the diuretics of this class is the collecting tubule of the kidney, where they interfere with sodium reabsorption and indirectly with potassium secretion. Their diuretic activity is weak because the fractional sodium reabsorption in the collecting tubule usually does not exceed 3% of the filtered load. Spironolactone is an example of a potassium sparing diuretic that is an aldosterone antagonist that acts as a competitive inhibitor of aldosterone to decrease sodium. This results in a decrease in potassium secretion.

Another common treatment for HF is the administration of angiotensin converting enzyme (ACE) inhibitors. ACE inhibitors produce a moderate increase in cardiac output and reduce the incidence of ventricular arrhythmias, without increasing the heart rate. ACE inhibitors are employed for the treatment of cardiac indications such as hypertension and heart failure. It is known that at least some ACE inhibitors can improve (decrease) morbidity and mortality in patient populations with heart conditions. The principal pharmacological and clinical effects of ACE inhibitors arise from suppressing the synthesis of angiotensin II by blocking the conversion of angiotensin I to angiotensin II. Blood pressure is lowered from inhibition of angiotensin II biosynthesis, especially where hypertension is angiotension II-related.

One of the advantages of ACE inhibitors in the management of heart disease is the low occurrence of adverse effects. A dry irritating cough is the most frequent side effect. ACE inhibitors do not adversely affect serum lipids, plasma glucose or uric acid, however, they tend to increase serum potassium in patients with chronic renal failure or in patients taking potassium-sparing diuretics, potassium supplements, or NSAIDs. Over the counter drugs such as NSAIDs and aspirin may complicate the administration of cardiac agents due to interference and other unwanted reactions. For instance, aspirin may reduce the effect of ACE inhibitors in HF, possibly because it inhibits the effects of kinins.

The treatment of heart failure is very complicated because there are many different etiologies for the disease, and consequently there are many treatment methods. Selecting the appropriate therapy can be a challenge to the practicing physician because a combined regimen of several different agents is required to manage patient symptoms and return the heart to normal. This presents the physician with a number of challenges. First, many of the agents used in combination may react with each other to cause side effects for the patient. In addition, certain agents may potentiate or negate the effect of agents given concurrently or concomitantly. Second, many agents are contraindicated for use together due to those interactions. Furthermore, the dose prescribed must maintain efficacy while not interfering with the action of the other agents being given concormitantly.

For the patient, managing the effects of the illness is complicated by a regimen of different agents. Although there are some agent combinations, most agents are administered in separate compounds and may even require administration at different times and at different frequencies. The symptoms of heart failure may cause patients to be quite weak and confused and remembering to follow the agent regimen can be a challenge. In addition, the side effects caused by some agents may exacerbate the disease being treated if the dosage is not carefully monitored. This often results in non-compliance, especially in the elderly, or in those who do not have access to professional care-givers.

Thus, there is a need for compositions and methods for treatment of cardiac indications, such as heart failure, and congestive heart failure, and hypertension, by the administration of compositions comprising a combination of at least two or more agents in an effective amount to treat the symptoms of cardiac indications. Such compositions and methods could provide a means to treat cardiac indications with several agents without administering multiple individual agents either concurrently or separately.

What is also needed are compositions and methods for treatment of cardiac indications by the administration of compositions comprising a combination of at least two or more agents that are easily used and increase compliance by patients, easily administered by caregivers, and that facilitate the physicians determination of agent interactions, patient side effects, and dosages. Preferably, such compositions and methods provide for oral administration.

### SUMMARY OF THE INVENTION

The present invention is directed to methods and compositions for treating cardiac indications in humans and animals, comprising combinations of active agents. Such active agents include, but are not limited to, therapeutically effective amounts of ACE inhibitors, loop diuretics, and potassium sparing diuretics. Preferably the combinations are in single compositions comprising at least two or more agents, or more preferably, three agents. Methods and compositions are provided for the treatment of heart failure, congestive heart failure, and other cardiac indications. The compositions can be formulated in the form of tablets, capsules, sterile solutions or suspensions, compounded in a conventional manner with physiologically acceptable vehicles or carriers, recipients, binders, preservatives, stabilizers, flavorings, or the like, as called for by accepted pharmaceutical practice.

Preferably, ACE inhibitors of the present invention can comprise caporal, enalapril, lisinopril, benazepril, fosinopril, quinapril, ramipril, the salts thereof, and other functional equivalents. Preferably, loop diuretics of the present invention can comprise furosemide, ethacrynic acid, bumetamide, the salts thereof, and other functional equivalents. Preferably, potassium sparing diuretics of the present invention can comprise spironolactone, triamterine, amiloride, the salts thereof, and functional equivalents.

The present invention comprises injectable and noninvasive routes for agent delivery, including but not limited to, the oral, nasal, pulmonary, rectal, buccal, vaginal, transdermal and ocular routes. Compositions comprising combinations of at least two or more agents may be administered through these routes of administration in compositions that allow for sustained release, controlled release or time-release dosing to the patient. The release profile of the compositions of the present invention allows for greater safety in administration of multiple agents, reduces the number of factors a physician must consider in treating cardiac indications with a multiple agent regimen, provides for greater compliance in patients, and results in fewer side effects for patients.

While studies have demonstrated a benefit of multi-drug therapy, the tasks of administering several different agents proves to be a challenge for the physician, patients, and caregivers. The present invention provides compositions and methods for treatment of cardiac indications by the administration of compositions comprising a combination of at least two or more agents in an effective amount to treat the symptoms of cardiac indications.

The present invention provides compositions and methods for administering compositions comprising combinations of two or more agents in compositions that are easily administered to persons having cardiac indications such as heart failure, congestive heart failure, and hypertension.

The present invention also provides compositions and methods for administering compositions comprising combinations of two or more agents that promote high patient acceptance and compliance in persons with cardiac indications such as heart failure and congestive heart failure, and hypertension.

The present invention provides compositions and methods for administering compositions comprising combinations of two or more agents that maximize agent absorption in persons having cardiac indications such as heart failure and congestive heart failure, and hypertension.

These and other features and advantages of the present invention will become apparent after a review of the following detailed description of the disclosed embodiments and the appended claims.

### DETAILED DESCRIPTION

The present invention is directed to methods and compositions for treatment of cardiac conditions. These conditions include, but are not limited to, hypertension, heart failure, congestive heart failure, and other cardiac indications. Preferred methods of treatment include the administration of compositions comprising combinations of at least two or more agents in an effective amount to treat symptoms of these conditions. Compositions comprise combinations of at least two or more agents comprising ACE inhibitors, loop diuretics, and potassium sparing diuretics. The methods of the present invention comprise routes of administration that include, but are not limited to, oral, buccal, nasal, transdermal, injectable, slow release, controlled release, iontophoresis, sonophoresis, and other delivery devices and methods. Injectable methods include, but are not limited to, parenteral routes of administration, intravenous, intramuscular, subcutaneous, intraperitoneal, intraspinal, intrathecal, intracerebroventricular, intraarterial and other routes of injection. The methods include frequency of administration that is dependent upon the patient condition, method of administration and concentration of the active agent.

The present invention comprises compositions that provide formulations for controlled, slow release, or sustained release of the therapeutic compounds over a predetermined period of time. Methods of administration of compositions comprising combinations of at least two or more agents using these formulations allow for a desired concentration of these agents to be maintained in the bloodstream of the patient for a longer period of time than with conventional formulations. Slow release, controlled or sustained release formulations are known to those skilled in the art and include formulations such as coated tablets, pellets, capsules, dispersion of the active agent in a medium that is insoluble in physiologic fluids or where the release of the active agent is released after degradation of the formulation due to mechanical, chemical or enzymatic activity, or is released from an implantable device.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting. Definitions

It must be noted that, as used in this specification and the appended claims, the singular forms “a,” “an,” and “the” include plural referents unless the context clearly dictates otherwise. Thus, for example, reference to a bilayer tablet containing “compositions comprising combinations of two or more agents” may include a mixture of such compositions, reference to “an adhesive” includes reference to one or more of such adhesives, and reference to “a bile salt” includes reference to a mixture of two or more of such bile salts.

The phrase “cardiac indications” includes patients who are at risk of suffering from this condition relative to the general population, even though they may not have suffered from it yet, by virtue of exhibiting risk factors. Cardiac indications includes any cardiac condition resulting from multiple etiologies.

The phrase “congestive heart failure” includes patients who are at risk of suffering from this condition relative to the general population, even though they may not have suffered from it yet, by virtue of exhibiting risk factors. For example, a patient with untreated hypertension may not have suffered from congestive heart failure, but is at risk because of the hypertensive condition.

The term “heart failure” includes patients who are at risk of suffering from this condition relative to the general population, even though they may not have suffered from it yet, by virtue of exhibiting risk factors.

The term “treating” “treatment” “treat” as used herein includes preventative, emergency, and long-term treatment.

The term “multi-agent compound” includes any compositions comprising combinations of at least two or more agents for administration to patients to treat medical conditions.

The term “ACE inhibitor” includes any agent used to treat cardiac indications by inhibiting the conversion of angiotensin I to angiotensin II.

The term “potassium sparing diuretic” as used herein refers to diuretics that do not deplete potassium.

The term “aldosterone antagonist” includes one group of potassium sparing diuretics that acts by inhibiting aldosterone.

The terms “drug”, “agent”, “active agent”, “medication”, and the like are considered to be synonymous and all refer to the component that has a physiological effect on the individual to whom the composition is administered.

As used herein, “chemical enhancer,” “penetration enhancer”, “permeation enhancer,” and the like shall be inclusive of all enhancers that increase the flux of a permeant, agent, or other molecule across the mucosa and is limited only by functionality. In other words, all cell envelope disordering compounds, solvents, steroidal detergents, bile salts, chelators, surfactants, non-surfactants, fatty acids, and any other chemical enhancement agents are intended to be included.

Permeation enhancers are comprised of two primary categories of components, i.e., cell-envelope disordering compounds and solvents or binary systems containing both cell-envelope disordering compounds and solvents. As discussed above, other categories of permeation enhancer are known, however, such as steroidal detergents, bile salts, chelators, surfactants, non-surfactants, and fatty acids.

Cell envelope disordering compounds are known in the art as being useful in topical pharmaceutical preparations and function also in agent delivery through the skin or mucosa. These compounds are thought to assist in dermal penetration by disordering the lipid structure of the stratum comeum cell-envelopes. A list of such compounds is described in European Patent Application 43,738, published Jun. 13, 1982, which is incorporated herein by reference. It is believed that any cell envelope disordering compound is useful for purposes of this invention.

Suitable solvents include water; diols, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl) pyrrolidone, N-methylpyrrolidone, 1-dodecylazacycloheptan-2-one and other n-substituted alkyl-azacycloalkyl-2-ones (azones) and the like.

As used herein, “bile salts” means steroidal detergents that are the natural or synthetic salts of cholanic acid, e.g. the salts of cholic and deoxycholic acid or combinations of such salts, and the unionized acid form is also included. Bile salt analogs having the same physical characteristics and that also function as permeation enhancers are also included in this definition.

As used herein, “transmucosal,” “transbuccal,” and similar terms mean passage of a multi-agent composition into and through the mucosa to achieve effective therapeutic blood levels or deep tissue levels.

As used herein, “effective amount” means an amount of a multi-agent composition that is sufficient to provide a selected effect and performance at a reasonable benefit/risk ratio attending any medical treatment. An effective amount of a permeation enhancer, as used herein, means an amount selected so as to provide the selected increase in permeability and, correspondingly, the desired depth of penetration, rate of administration, and amount of agent delivered.

As used herein, “single administrative dose” means that the agents are combined in a composition that is provided to the individual in one administration. The individual agents are provided in the composition at the desired concentration for each agent and the composition may be administered as many times a day to the patient as is necessary.

As used herein, “adhesive,” “adhesive polymer”, “mucoadhesive”, or such similar terms refers to hydrophilic polymers, natural or synthetic, which, by the hydrophilic designation, can be either water soluble or swellable and which are compatible with the enhancers and compositions comprising two or more agents combined. Such adhesives function for adhering the dosage forms to the mucous tissues of the oral cavity, such as the gingiva. Such adhesives are inclusive of hydroxypropyl cellulose, hydroxypropyl methylcellulose, hydroxy ethylcellulose, ethylcellulose, carboxymethyl cellulose, dextran, guar gum, polyvinyl pyrrolidone, pectins, starches, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, polyethers, and mixtures thereof and the like.

By “system”, “drug delivery system”, “transmucosal delivery system” or the like is meant a unit dosage form of a drug or agent composition, preferably any compositions comprising combinations of at least two or more agents, including carriers, enhancers, and other components, in which the multi-agent compound is contained in or accompanied by means for maintaining the drug composition in a drug transferring relationship or providing any multi-agent compounds to the desired site in the body. Such means can be either a patch, tablet, troche, or other device of determined physical form for continuous agent administration thereto for systemic transport, or such means can be formulated in free form to be applied directly to the patient as a cream, gel, gum, ointment and the like.

The term “troche” includes pastille, lozenge, morsulus, rotula, trochiscus, and the like.

“Free form” means that the formulation is spreadable or malleable into a selected shape at the time of application.

“Determined physical form” means that the formulation has a form determined by a device. The means used may be a device such as a tablet or matrix patch or liquid reservoir patch. A matrix patch contains the agent, permeation enhancer, and other optional ingredients suspended or dispersed in an adhesive layer. A reservoir patch contains the agent, permeation enhancer, and other optional ingredients in a reservoir, which can be in liquid form, or the liquid can be gelled or thickened by an agent such as mineral oil, petroleum jelly and various aqueous gelling agents and hydrophilic polymers. Such a reservoir or matrix patch is brought into contact with the surface and is held in place by a suitable adhesive. In a reservoir patch, the agent composition is applied to the surface through a permeable membrane forming the reservoir floor that is in direct contact with the surface.

A preferred aspect of the present invention can comprise combinations of at least two or more agents, such as for instance, ACE inhibitors, loop diuretics, and potassium sparing diuretics. When a diuretic and an ACE inhibitor are administered together, the pharmacological action of one agent will counteract the effect of the other because diuretics and ACE inhibitors have different effects on the renin-angiotensin-aldosterone system (RAAS). Diuretics regulate the sodium-balance and fluid volume and decrease both sodium and fluid volume following therapy. This results in an increase in plasma renin activity, activates the renin-angiotensin-aldosterone system (RAAS) which increases the conversion of angiotensin I to angiotensin II to counteract the blood pressure lowering effect of the diuretic. ACE inhibitors act by inhibiting the conversion of angiotensin I to angiotensin II, potentiating the blood pressure lowering effect of the diuretic. The dose of a coadministered diuretic may frequently be reduced, especially if ACE inhibitor-induced renal insufficiency occurs. It is possible to establish the highest non-pharmacological active dose of diuretic, i.e. a dose that is so low that it has no effect on blood pressure, and no apparent adverse effects. The highest non-effective dose of diuretic will still trigger the renin-angiotensisn-aldosterone system and although it has no physiological effect of it's own, it will nonetheless have a potentiating effect on an ACE inhibitor.

Mortality in heart failure is decreased when patients are treated with agents such as loop diuretics, ACE inhibitors, and potassium sparing diuretics such as spironolactone. It appears that residual aldosterone mediates many harmful effects in chronic heart failure and that to optimize the benefit of blocking the renin-angiotensin-aldosterone system may require specific blockade of residual aldosterone as well as traditional angiotensin-converting enzyme inhibition. As a result, treatment with spironolactone is useful when administered with loop diuretics and an angiotensin-converting enzyme (ACE) inhibitor in severe congestive heart failure (CHF) because spironolactone is an aldosterone antagonist.

The Randomized Aldactone Evaluation Study (RALES) found that spironolactone (Aldactoneg®, Searle) administered along with captopril (CapotenV®, Bristol Myers Squibb) and furosemide (Lasix®, Roche) reduced morbidity and mortality in patients with severe HF by 27%. (Pitt, B, et al, 1999, The effect of Spironolactone on morbidity and mortality in patients with severe heart failure. NEJM; 341: 709-717.) The RALES investigators concluded that the addition of an aldosterone antagonist to the maintenance treatment of an ACE inhibitor and loop diuretic is safe and effective in many patients with CHF. Addition of at least a third agent to the treatment of HF only complicated the administration of such treatment, because the patients now had at least three different agent schedules to meet. Additionally, there was no attempt to administer combinations of agents that did cause unnecessary side effects.

A study by MacFadyen reported in Cardiovasc Res 1997 July; 35(1):30-4 showed that in a placebo-controlled, double-blind study of 31 patients with stable Congestive Heart Failure (NYHA class II-IV), treatment with spironolactone lowered morbidity. Patients with stable chronic heart failure were treated with spironolactone (50-100 mg/day) or placebo in addition to diuretics and ACE inhibitor. Subjects who received spironolactone in addition to diuretics and ACE inhibitors for 8 weeks of active therapy, showed approximately 20% reduction in myocardial fibrosis activity. The study reported reduced vascular collagen turnover, improved heart rate variability and reduced early morning rise in heart rate in heart failure patients treated with spironolactone. Other studies have indicated similar results when spironolactone therapy is added to the treatment regimen of cardiac patients undergoing therapy consisting of a loop diuretic and an ACE inhibitor. See Kinugawa, et al. Gen Pharmacol 1998 July; 31(1):93-9; Struthers, J Card Fail 1996 March; 2(1):47-54; , Barr C S, et al., Am J Cardiol 1995 December 15; 76(17):1259-65; Han Y L et al., Chin Med J (Engl)1994 September; 107(9):688-92.

A study in which angiotensin-converting enzyme inhibitor and spironolactone were used for the treatment of Congestive Heart Failure appeared in Am J Cardiol Zannad F, Jan. 21, 1993; 71(3):34A-39A The study contended that secondary aldosteronism has deleterious effects in patients with CHF and can contribute to congestion, ventricular arrhythmias, and sudden death. It was known that mortality occurs more frequently in patients with elevated levels of plasma aldosterone, which increased as CHF progressed as a result of activation of the renin-angiotensin-aldosterone system (RAAS). This was further amplified by the routine use of diuretics. The study proposed that using both an ACE inhibitor and spironolactone achieved a more complete inhibition of the whole RAAS and may produce further clinical benefits. However, the efficacy and safety of such a combination were not addressed in this study and such issues should be addressed in a future study. Other studies have indicated use of spironolactone and ACE inhibitor therapies for treatment of cardiac disease. See lkram H et al., Aust N Z J Med 1986 February; 16(1):61-3.

The present invention comprises compositions and methods of administering compositions comprising combinations of at least two or more agents for the treatment of cardiac indications. The compositions of the present invention preferably comprise ACE inhibitors, loop diuretics, and potassium sparing diuretics in compositions comprising combinations of at least two or more agents. The present invention also comprises the use of other forms of ACE inhibitors, loop diuretics and potassium sparing diuretics. In addition, the compositions of the present invention comprise delivery vehicles or permeation enhancers known to those skilled in the art.

The compositions of the present invention may further include pharmaceutically acceptable carriers. The compositions may also include other medicinal agents, pharmaceutical agents, carriers, adjuvants, diluents and other pharmaceutical preparations known to those skilled in the art. Such agents are known to those skilled in the art and are generally described as being biologically inactive and can be administered to patients without causing deleterious interactions with the active agent. Examples of carriers or excipients for oral administration include corn starch, lactose, magnesium stearate, microcrystalline cellulose and stearic acid, povidone, dibasic calcium phosphate and sodium starch glycolate. Any carrier suitable for the desired administration route is contemplated by the present invention.

A preferred aspect of the present invention comprises compositions and methods of administration of compositions comprising combinations of ACE inhibitors and loop diuretics in a single administrative dose. A more preferred aspect of the present invention comprises compositions comprising combinations of ACE inhibitors, loop diuretics and potassium sparing diuretics in a single administrative dose. An even more preferable aspect of the invention comprises compositions comprising loop diuretics and potassium sparing diuretics in a single administrative dose. A most preferred aspect comprises a composition comprising combinations of ACE inhibitors and potassium sparing diuretics in a single administrative dose.

For example, a preferred aspect of the present invention comprises compositions comprising combinations of captopril and furosemide in a single administrative dose. A more preferred aspect of the present invention comprises compositions comprising combinations of captopril, furosemide, and spironolactone in a single administrative dose. An even more preferable aspect of the present invention comprises compositions comprising furosemide and spironolactone in a single administrative dose. A most preferred aspect comprises compositions comprising combinations of captopril and spironolactone in a single administrative dose.

The routes of administration for agents is chosen according to the speed of absorption desired and the site of action of the agent. Some agents are formulated for a specific route only and must be given in that manner. Various routes of administration of the present invention are presented herein.

Oral and enteral administration require that the agent not be destroyed by the environment of the stomach and digestive enzymes. This means is too slow if rapid absorption is required, and cannot be used if the patient is vomiting. Rectal administration in the form of liquids or suppositories circumvents this problem in enteral administration. Rectal suppositories can be prepared by mixing the agent with a suitable non-irritable vehicle, for example, cocoa butter and polyethylene glycol, which is in the solid state at ordinary temperatures, in the liquid state at temperatures in intestinal tubes and melts in the rectum to release the agent.

Mucosal routes of administration other than the above include absorption through the nasal mucosa, the buccal mucosa, sublingually, or the bronchioles, the latter usually achieved through inhalation of an aerosol. Vaginal or rectal administration are also mucosal routes of agent.

Percutaneous administration is used for iontophoresis or by direct absorption through the skin. Iontophoreses is the electrically driven application of agents or medicants, in their ionic form, to the surface tissues of a patient. The application of electric current causes migration of ions into the tissue wherein such migration is proportional to the quantity of current applied through the iontophorectic system. Direct absorption can be from application of the agent to the skin surface by means of a cream.

Parenteral administration is used when an agent cannot be given by mouth. The speed of absorption varies greatly with the specific route used, which may be subcutaneous, intravenous, intramuscular, intra-arterial, intraperitoneal, intrathecal, intracardiac, or intrastemal.

The rate of absorption of an agent administered as a tablet or other solid oral-dosage form is partly dependent upon its rate of dissolution in the gastrointestinal fluids. This factor is the basis for the so-called controlled-release, extended release, sustained-release, or prolonged-action pharmaceutical preparations that are designed to produce slow, uniform absorption of the agent for 8 hours or longer. Potential advantages of such preparations are reduction in the frequency of administration of the agent as compared with conventional dosage forms resulting in improved compliance by the patient, maintenance of a therapeutic effect overnight, and decreased incidence and or intensity of undesired effects by elimination of the peaks in drug concentration that often occur after administration of immediate-release dosage forms.

The methods of administration of the present invention can vary within limits, but necessarily involve providing the selected compositions comprising combinations of at least two or more agents to the patient such that drug delivery is initiated and continues for a period of time sufficient to provide the selected pharmacological or biological response. The frequency of administration of treatment depends upon the patient condition, mode of delivery and concentration of active agent. Cardiac treatment can be delivered as often as needed (ql), four times daily (qid), daily (qd) or at certain times in a 24 hour cycle such as after eating or at bedtime.

Simple multi-agent compound agent delivery systems of the present invention comprise capsules containing differently coated pellets of the agent. On release from the capsule, the uncoated pellets provide an initial amount of the composition comprising the combination of two or more agents to the body, and the coated pellets provide the multi-agent composition over a period of time. Another system comprises a tablet made from a polymer containing the multi-agent compound dispersed within. As the polymer slowly degrades in the stomach, the multi-agent compound is released. Additional agent delivery systems include hydrogel materials with coated pills embedded in the hydrogel, such as that taught in U.S. Pat. No. 4,659,558. The unswollen hydrogel is swallowed and in the presence of fluids in the stomach, swells so that the hydrogel is retained within the stomach. The coated pills are released as the hydrogel degrades.

The present invention comprises methods of administering compositions comprising combinations of at least two agents, preferably including ACE inhibitors, loop diuretics and potassium sparing diuretics, in a single administrative dose for treatment of cardiac conditions. The dosages of the multi-agent compositions administered depend on the condition being treated, the particular composition, and other clinical factors such as weight and condition of the human and the route of administration of the compositions. Preferable amounts of ACE inhibitors are administered in a range of between about 0.5 mg/day and about 500 mg/day, more preferably between about 5 mg/day and about 100 mg/day and most preferably between about 25 mg/day and about 50 mg/day. Preferable amounts of potassium sparing diuretics are administered in a range of between about 0.5 mg/day and about 500 mg/day, more preferably between about 5 mg/day and about 100 mg/day and most preferably between about 12.5 mg/day and about 25 mg/day. Preferable amounts of loop diuretics are administered in a range of between about 0.5 mg/day and about 500 mg/day, preferably between about 5 mg/day and about 100 mg /day and most preferably between about 40 mg/day and about 80 mg/day.

A preferred aspect of the present invention comprises methods of administering cardiac effecting agents such as ACE inhibitors, loop diuretics and potassium sparing diuretics in compositions comprising a combination of at least two or more agents in a single administrative dose. In a preferred aspect, the composition comprising the combination of at least two or more agents is administered whenever needed (ql). In a more preferred aspect, the composition comprising the combination of at least two or more agents is administered four times daily (qid). In a most preferred aspect, the composition comprising the combination of at least two or more agents is administered daily (qd).

A preferred aspect of the present invention comprises compositions comprising combinations of captopril in a concentration of between about 25 mg/day to about 50 mg/day, and furosemide in a concentration of between about 40 mg/day to about 80 mg/day in a single administrative dose. A more preferred aspect of the invention comprises compositions comprising combinations of captopril in a concentration of between about 25 mg/day to about 50 mg/day, furosemide in a concentration of between about 40 mg/day to about 80 mg/day, qd, and spironolactone in a concentration of between about 12.5 mg/day and about 25 mg/day, in a single administrative dose. An even more preferable aspect of the invention comprises compositions comprising the loop diuretic furosemide in a concentration of between about 40 mg/day to about 80 mg/day, and spironolactone in a concentration of between about 12.5 mg/day and about 25 mg/day, in a single administrative dose. A most preferred aspect comprises compositions comprising captopril in a concentration of between approximately 25 mg/day to 50 mg/day, and spironolactone in a concentration of approximately between 12.5 mg/day and 25 mg/day, in a single administrative dose.

Preferred methods of administration of compositions comprising combinations of at least two or more agents in a single administrative dose include oral routes. The compositions of the present invention can be contained in a gelatin capsule, tablet, liquid or powder, and such items may be coated for ease of swallowing. For oral administration, fine powders or granules may contain diluting, dispersing, and or surface active agents and may be present in water or in a syrup, in capsules or sachets in the dry state, or in a nonaqueous solution or suspension wherein suspending agents may be included, in tablets wherein binders and lubricants may be included or in a suspension in water or a syrup. Components that may be added such as flavoring, preserving, suspending, thickening or emulsifying agents. Such preparations are known or apparent to those skilled in the art.

One aspect of the present invention comprises methods of treatment of cardiac indications such as heart failure and congestive heart failure comprising administration of compositions comprising a combination of at least two or more agents in a single administrative dose through oral delivery compositions and devices. Oral administration includes, but is not limited to, administration through the mucosa of the mouth and any other surfaces of the alimentary canal, stomach, and the gastrointestinal tract. Oral delivery methods are often limited by chemical and physical barriers imposed by the body, such as the varying pH in the gastrointestinal tract, exposure to enzymes and the impermeability of the gastrointestinal membranes. Methods of the present invention for orally administering multi-agent compositions may also include the coadministration of adjuvants with the compositions of the present invention. For example, resorcinols and nonionic surfactants such as polyoxyethylene oleyl ether and n-hexadecyl polyethylene ether, can be administered with or incorporated into the compositions of the present invention to artificially increase the permeability of the intestinal walls. Other methods include the coadministration of enzymatic inhibitors with the compositions of the present invention. Liposomes and emulsions are also contemplated in the present invention for delivery of the compositions.

Methods of treatment of the present invention comprise administration of compositions comprising combinations of at least two or more agents in a single administrative dose using microspheres of artificial polymers or proteins that are used for delivery of compositions through various routes, such as gastrointestinal or nasal. Nasal delivery is considered an efficacious route of administration for treatment of cardiac indications such as heart failure and congestive heart failure because the nose has a large surface area available for agent absorption due to the coverage of the epithelial surface by numerous microvilli and the subepithelial layer is highly vascularized. The venous blood from the nose passes directly into the systemic circulation and avoids the loss of agent in a first pass metabolism in the liver.

In order to enhance nasal delivery, absorption enhancers can be added to the compositions of the present invention. Bile salts or derivatives such as fusidic acid, or surfactants, especially nonionic surfactants, can be used to modify the properties of the nasal mucosa to enhance uptake. Microspheres can also be used, particularly those that swell in the presence of moisture. Albumin, starch and DEAE-Sephadex microspheres of 40-60 μm in diameter have been used. These same absorption enhancers can be used in the present invention for enhanced absorption across other mucosal surfaces, such as the gastrointestinal tract or the oral cavity.

Other methods of the present invention comprise treatment of cardiac indications such as heart failure and congestive heart failure by administration of cardiac effecting agents such as ACE inhibitors, loop diuretics and potassium sparing diuretics in compositions comprising combinations of at least two or more agents in a single administrative dose through the buccal and sublingual membranes. Both the buccal and sublingual membranes offer advantages over other routes of administration. For example, compositions administered through the buccal and sublingual routes have a rapid onset of action, reach high levels in the blood, avoid the first-pass effect of hepatic metabolism and avoid exposure of the multi-agent composition to fluids of the gastrointestinal tract. Additional advantages include easy access to the membrane sites so that the multi-agent compositions can be applied, localized and removed easily. Further, there is good potential for prolonged delivery through the buccal membrane. Administration through the buccal mucosa may be better accepted than rectal dosing and generally avoids local toxic effects, such as has been a problem in nasal administration.

The sublingual mucosa includes the membrane of the ventral surface of the tongue and the floor of the mouth, whereas the buccal mucosa constitutes the lining of the cheek and lips. The sublingual mucosa is relatively permeable, thus giving rapid absorption and acceptable bioavailabilities of many agents. Further the sublingual mucosa is convenient, easily accessible, and generally well accepted. This route has been a traditional route of administration of nitroglycerin and also buprenorphine and nifedipine. The sublingual mucosa is not well suited to sustained-delivery systems because it lacks an expanse of smooth and relatively immobile mucosa suitable for attachment of a retentive delivery system.

Solutes that facilitate the transport of solutes across biological membranes, known as penetration or permeation enhancers, are well known in the art for administering agents. Such compositions are contemplated by the present invention as members of embodiments of the multi-agent compositions. Penetration enhancers can be categorized as chelators, e.g., EDTA, citric acid, and salicylates; surfactants, such as sodium dodecyl sulfate (SDS); non-surfactants, e.g., unsaturated cyclic ureas; bile salts, e.g., sodium deoxycholate, sodium taurocholate; and fatty acids e.g., oleic acid, acylcamitines, mono- and diglycerides.

Penetration enhancers are effective in facilitating mucosal agent administration. For an enhancer to work effectively, the enhancer and multi-agent composition combination is held in position against mucosal tissues for a period of time sufficient to allow enhancer-assisted penetration of the ACE inhibitors, loop diuretics and spironolactone multi-agent composition across the mucosal membrane. In transdermal and transmucosal technology, this is often accomplished by means of a patch or other device that adheres to the skin layer by means of an adhesive.

One of the agents that can be included in the pharmaceutical composition is a permeation enhancer. A permeation enhancer allows for more penetration of the active agents through the mucous membranes of the body. Permeation enhancers may also be incorporated in transdermal delivery systems. A permeation enhancer is preferably a member selected from the group consisting of cell envelope disordering compounds, solvents, steroidal detergents, bile salts, chelators, surfactants, non-surfactants, fatty acids, and mixtures thereof. A preferred organic solvent is a member selected from the group consisting of a C, or C3 alcohol, and C3 or C4 diol, DMSO, DMA, DMF, 1-n-dodecylcyclazacyclo-heptan-2-one, N-methyl pyrrolidone, N-(2hydroxyethyl) pyrrolidone, triacetin, propylene carbonate and dimethyl isosorbide and mixtures thereof. A preferred cell-envelope disordering compound is a member selected from the group consisting of isopropyl myristate, methyl laurate, oleic acid, oleyl alcohol, glycerol monoleate, glycerol dioleate, glycerol trioleate, glycerol monostearate, glycerol monolaurate, propylene glycol monolaurate, sodium dodecyl sulfate, and sorbitan esters and mixtures thereof. A preferred bile salt is a steroidal detergent selected from the group consisting of natural and synthetic salts of cholanic acid and mixtures thereof.

Oral adhesives are well known in the art. These adhesives consist of a matrix of a hydrophilic, water soluble or swellable, polymer or mixture of polymers that can adhere to a wet mucous surface. These adhesives may be formulated as ointments, thin films, tablets, troches, and other forms. These adhesives may have multi-agent compositions mixed therewith to effectuate slow release or local delivery of a multi-agent composition. Some have been formulated to permit absorption through the mucosa into the circulatory system of the individual.

Another delivery system that is contemplated by the present invention is the controlled released system. The benefits of controlled release delivery systems for delivery of the compositions of the present invention are significant, and provide for reduction in the number of doses and steady drug levels in the blood. One type of agent delivery system comprises using compositions that remain in the stomach over a prolonged period of time. The agent delivery system remains in the stomach and acts as an in vivo reservoir that releases agent at a controlled rate and continuously for absorption in the stomach or for passage to the intestines for absorption. Often the agent is administered from a delivery system that releases a agent as the system moves through the gastrointestinal tract over time. These systems eliminate the need for administering a number of single doses at periodic intervals. This system also provides the advantage of continuously supplying agents so that the blood levels of the agent are controlled and remains at an optimum level.

In controlled release systems contemplated in the present invention, after oral ingestion, agents are released by diffusion and erosion throughout the gastrointestinal tract to a significant degree. Methods of the present invention for the prolongation of gastric retention time, include incorporation of fatty acids to reduce physiological gastric emptying and the use of bioadhesive polymers. Such systems are known to those skilled in the art and comprise using polymers such as polycarbophyll, sodium carboxymethylcellulose, tragacanth gum, acrylates and methacrylates, modified celluloses and polysaccharide gums.

Another delivery system that is contemplated by the present invention for targeting agents to the stomach while avoiding gastric emptying is known as a hydrodynamically balanced system. This system is based on capsules or tablets with bulk density lower than gastric fluid. Thus, the dosage form stays buoyant in the stomach. These dosage forms are comprised of 20-75% of one or more hydrocolloids, e.g., hydroxyethylcellulose and hydroxypropylmethylcellulose.

Other methods of delivery include gastroinflatable delivery devices. These devices contain one or several inflatable chambers that are filled with gas at body temperature by a gasifying liquid or a gas-forming solid, such as bicarbonate or carbonate. The chambers are incorporated within a plastic matrix and the whole structure is encapsulated in gelatin. Dissolution of the gelatin coating inflates the device and agent diffusion occurs.

Other types of these devices include osmotic pressure compartments containing osmotically active salts. In the present invention, dissolution of these salts by the gastric fluid pumps out the ACE inhibitors, loop diuretics and spironolactone multi-agent composition. Others are based upon a floating bilayer compressed matrix. One of the layers is comprised of a hydrophilic polymer and a carbon dioxide generating composition. The carbon dioxide maintains buoyancy and the other hydrophilic layer releases the agent from the matrix. A further method for gastric agent targeting involves an intragastric retention shape, made of polyethylene or polyethylene blend. The delivery systems described above may also be used in the present invention to target multi-agent compositions to the upper small intestine. However targeting to other areas of the small intestine may involve several additional systems.

The low stomach pH and presence of gastric enzymes have led to the development of enteric coating. This coating protects the gastric mucosa from agent irritation. Coating is done with a selectively insoluble substance, and protects agents from inactivation by gastric enzymes and/or low pH. The most common enteric coatings are methacrylic acid copolymers, cellulose acetate phthalate, cellulose acetate succinate, and styrol maleic acid copolymers. The most significant drawback of enteric coating is the variability in gastric emptying time. This results in a large variance in blood agent levels.

Another method of drug delivery in the small intestine comprises delivery systems that allow for agent absorption via the lymphatic system. Capillary and lymphatic vessels are permeable to lipid-soluble compounds and low molecular weight moieties. Another approach for targeting agents to the small intestine involves the use of intestinal sorption promoters. Such promoters include long chain fatty acids, including linoleic acid, acylcamitines, and palmitocamitine. Bioadhesives can also be used in the present invention to prolong intestinal transit, as in buccal delivery systems. The adhesion to the intestinal mucosa takes place either by mechanical interlocking or other mechanisms.

A preferred tablet for oral administration in the methods of the present invention, preferably for buccal delivery systems, comprises an adhesive layer comprising a hydrophilic polymer with one surface adapted to contact a first tissue of the oral cavity and adhere thereto when wet and an opposing surface in contact with and adhering to an adjacent agent/enhancer layer comprising a permeation enhancer and multi-agent composition. The agent/enhancer layer contacts and is in agent transfer relationship with the buccal mucosa when the adhesive layer contacts and adheres to the first tissue, preferably the gingiva. Preferably the hydrophilic polymer comprises compounds selected from the group consisting of hydroxypropyl cellulose, hydroxypropyl methylcellulose, hydroxyethylcellulose, ethylcellulose, carboxymethyl cellulose, dextran, guar-gum, polyvinyl pyrrolidone, pectins, starches, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, polyethers, and mixtures thereof. The adhesive layer may additionally contain one or more members selected from the group consisting of fillers, tableting excipients, lubricants, flavors, and dyes and that the agent/enhancer layer additionally contain one or members selected from the group consisting of tableting excipients, fillers, flavors, taste-masking agents, dyes, stabilizers, enzyme inhibitors, and lubricants.

The present invention also comprises delivery of compositions comprising combinations of at least two or more agents comprising ACE inhibitors, loop diuretics and potassium sparing diuretics in a single administrative dose to the colon. Because of its location at the distal portion of the alimentary canal, the colon is particular difficult to access. Enteric coatings have been used to bypass absorption in the stomach and deliver the agent to the small intestine. Delivery is based upon the pH differences between these two parts of the ahmentary canal. In current techniques for targeting agents to the colon, solid formulations of the desired agent molecules are coated with a pH-resistant polymeric coating. Such formulations are similar to enteric coated formulations which may be used to deliver agents to the distal ileum. Enteric coatings include bioerodible polymers such as shellac and cellulose acetate phthalate. Excipients such as triethanolamine myristate can be used for prolongation of GI transit time.

In contrast to the enteric-coated formulations, however, the formulations for colonic delivery are designed to withstand both low and slightly basic pH values for several hours. During this time, they are assumed to pass the stomach and the small intestine and reach the large intestine, where the coat disintegrates and the agent release process is initiated. The polymers used for this purpose are commonly acrylic acid derivatives or cellulose derivatives such as cellulose acetate phthalate or ethyl cellulose.

The present invention comprises methods of administration of compositions comprising combinations of at least two or more agents comprising ACE inhibitors, loop diuretics and potassium sparing diuretics in a single administrative dose in transdermal delivery systems for the treatment of heart failure and congestive heart failure. Transdermal methods provide methods of administration that have high patient compliance. The present invention comprises methods of treating heart failure and congestive heart failure that include transdermal patches or assisted transdermal delivery such as with electricity or ultrasound.

Transdermal drug delivery (TDD) offers several advantages over traditional delivery methods including injections and oral delivery. When compared to oral delivery, TDD avoids gastrointestinal agent metabolism, reduces first-pass liver metabolism effects, and provides sustained release of multi-agent compositions. In actuality, transdermal delivery is the transport of therapeutic compositions across the epidermis where the compositions get absorbed in the blood capillaries. When compared to injections, TDD eliminates the associated pain and the possibility of infection. The transdermal route of administration provides an alternative method and avoids gastrointestinal degradation and gastrointestinal uptake problems.

One detriment of transdermal delivery of therapeutic compositions is the low permeability of skin. This low permeability is attributed to the stratum comeum, the outermost skin layer which consists of dead cells and keratin fibers, keratinocytes, surrounded by lipid bilayers. The highly ordered structure of the lipid bilayers confers an impermeable character to the skin. The transdermal methods of the present invention include compositions of chemical, permeation or penetration enhancers and and methods of applying electricity or ultrasound to enhance transdermal multi-agent composition transport.

Ultrasound has been shown to enhance transdermal transport of agents (molecular weight less than 500) across human skin, a phenomenon referred to as sonophoresis. It has been shown that application of ultrasound at therapeutic frequencies (1 MHz) induces growth and oscillations of air pockets present in the keratinocytes of the skin in a process known as cavitation. These oscillations disorganize the skin lipid bilayers and enhance transdermal transport.

Transdermal agent delivery offers an advantageous alternative to oral delivery and injections. A variety of delivery systems can be used to enhance transdermal transport of agents. These include use of chemicals to either modify the skin structure or to increase the agent concentration in the transdermal patch; ii) applications of electric fields to create transient transport pathways, such as electroporation, or to increase the mobility of charged agents through the skin, such as in iontophoresis, and application of ultrasound, sonophoresis. U.S. Pat. No. 4,309,989 to Fahim and U.S. Pat. No. 4,767,402 to Kost, et al., disclose various ways in which ultrasound has been used to achieve transdermal agent delivery.

The present invention contemplates the administration of multiagent compositions provided in a single administrative dose using sonophoresis. Although a variety of ultrasound conditions have been used for sonophoresis, the most commonly used conditions correspond to the therapeutic ultrasound having frequency in the range of 1 MHz-MHz and intensity in the range of 0-2 W/cm². An optimal selection of ultrasound parameters, such as frequency, pulse length, intensity, as well as of nonultrasonic parameters, such as ultrasound coupling medium, can be conducted to ensure a safe and effeicacious application using the methods known in the art, such as are taught in U.S. Pat. No. 5,814,599, included herein in its entirety. For example, a preferred delivery method of the present invention uses ultrasound at a frequency of between 20 kHz and 10 kHz at an intensity that does not cause irreversible skin damage for a period of time effective to deliver the agent.

As used herein, sonophoresis is the application of ultrasound to the skin on which a multi-agent composition, alone or in combination with a carrier, penetration enhancer, lubricant, or other pharmaceutically acceptable agent for application to the skin, has been applied. Ultrasound is defined as sound at a frequency of between 20 kHz and 10 MHz, with intensities of between greater than 0 and 3 W/cm². As used herein, “low frequency” sonophoresis is ultrasound at a frequency that is less than 1 MHz, more typically in the range of 20 to 40 kHz, which is preferably applied in pulses, for example, 100 msec pulses every second at intensities in the range of between zero and 1 W/cm, more typically between 12.5 mW/cm² and 225 mW/cm². Exposures are typically for between 1 and 10 minutes, but may be shorter and/or pulsed. The intensity should not be so high as to raise the skin temperature more than about one to two degrees Centigrade.

Many ultrasound devices are available commercially which can be used in the present invention. For example, the ultrasonic devices used by dentists to clean teeth have a frequency of between about 25 and 40 kHz. Commercially available portable ultrasound toothbrushes make use of a small sonicator contained within the toothbrush. This sonicator is portable and operates on rechargeable batteries. Small pocket-size sonicators carried by patients and used to “inject” a therapeutic composition whenever required could be readily adapted from these devices.

The present invention comprises compositions comprising combinations of at least two or more agents comprising ACE inhibitors, loop diuretics and potassium sparing diuretics in a single administrative dose for treatment of cardiac indications such as heart failure and congestive heart failure. Not all administration routes are efficacious for every patient. Therefore, the present invention comprises various differing formulations of the ACE inhibitors, loop diuretics and potassium sparing diuretics as multi-agent compositions provided in single administrative doses. The formulations include those suitable for oral, rectal, ophthalmic, (including intravitreal or intracameral) nasal, topical (including buccal and sublingual), vaginal or parenteral (including subcutaneous, intramuscular, intravenous, intradermal, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into associate the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tamports, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as herein above recited, or an appropriate fraction thereof, of the administered ingredient.

It should be understood that in addition to the ingredients, particularly mentioned above, the formulations of the present invention may include other agents conventional in the art having regard to the type of formulation in question, for example, those suitable for oral administration may include flavoring agents. Many variations of the present invention may suggest themselves to those skilled in the art in light of the above detailed disclosure. All such modifications are within the full intended scope of the appended claims.

1. A composition to treat cardiac indications, comprising, a combination comprising at least two or more agents selected from an ACE inhibitor, a loop diuretic, and a potassium sparing diuretic, wherein the at least two or more agents are provided in a single administrative dose.

2. The composition of claim 1, wherein the combination comprises an ACE inhibitor and a potassium sparing diuretic.

3. The composition of claim 2, wherein the ACE inhibitor is captopril and the potassium sparing diuretic is spironolactone.

4. The composition of claim 1, wherein the combination comprises an ACE inhibitor, a loop diuretic, and a potassium sparing diuretic.

5. The composition of claim 4, wherein the ACE inhibitor is captopril, the loop diuretic is furosemide, and the potassium sparing diuretic is spironolactone.

6. The composition of claim 1, wherein the combination comprises an ACE inhibitor and a loop diuretic.

7. The composition of claim 6, wherein the ACE inhibitor is captopril and the loop diuretic is furosemide.

8. The composition of claim 1 is a method of treating cardiac indications.

9. The composition of claim 1 is the method of 11, wherein the effective amount of ACE inhibitor comprises a range of between about 500 mg/day.

10. The composition of claim 1 is the method of claim 11 wherein the effective amount of ACE inhibitor comprises a range of between about 5 mg/day and about 100 mg/day

11. A method of treating cardiac indications comprising:
administering an effective amount of a composition suitable for administration to an animal comprising at least two or more agents selected from an ACE inhibitor, a loop diuretic, and a potassium sparing diuretic, wherein the at least two or more agents are provided in a single dose.

12. The method of claim 11, wherein the effective amount of ACE inhibitor comprises a range of between about 0.5 mg/day and about 500 mg/day; the effective amount of loop diuretic comprises a range of between about 0.5 mg./day and about 500 mg/day; and the effective amount of potassium sparing diuretic comprises a range from between about 0.5 mg/day and about 500 mg/day.

13. The method of claim 11, wherein the effective amount of ACE inhibitor comprises a range of between about 5 mg/day and about 100 mg.day; and the effective amount of potassium sparing diuretic comprises a range from between about 5 mg/day and about 100 mg/day.

14. The method of claim 11, wherein the effective amount of ACE inhibitor comprises a range of between about 25 mg/day and about 50 mg/day; the effective amount of the loop diuretic comprises a range of between about 40 mg/day and about 80 mg/day; and the effective amount of potassium sparing diuretic comprises a range from between about 12.5 mg/day.

15. The method of claim 11 further comprising administering the composition by a route selected from oral, enteral, percutaneous, or parenteral.

16. The method of claim 11 further comprising administering the composition at least once a day.

17. The method of claim 11, wherein the cardiac indication is heart failure.

18. The method of claim 11, wherein the cardiac indication is hypertension.

19. A method of manufacturing a composition for treating cardiac indications comprising:
a) combining at least two agents selected from an ACE inhibitor, a diuretic, and a potassium sparing diuretic;
b) combining a pharmaceutical carrier with the combination of step (a).

20. The method of claim 19, wherein the unit dosage form is for oral administration.
